(19) United States
(12) Patent Application Publication
(10) Pub. No.: US 2025/0134831 A1
(43) Pub. Date: May 1, 2025

(54) Nasal Spray Compositions to Suppress Virus and Bacterial Pathogens and Methods of Use

(71) Applicant: Michael Anderson, Provo, UT (US)

(72) Inventor: Michael Anderson, Provo, UT (US)

(21) Appl. No.: 18/924,892

(22) Filed: Oct. 23, 2024

Related U.S. Application Data
(60) Provisional application No. 63/546,084, filed on Oct. 27, 2023.

(51) Int. Cl. A61K 31/05; A61K 9/00

(52) U.S. Cl. CPC A61K 31/05; A61K 9/0043

(57) ABSTRACT

A formulation of an antibacterial nasal composition effective in reducing viable pathogens and that is effective as preventive and adjuvant therapy for nasal infections or diseases caused by other pathogens.

### RELATED APPLICATION

This application claims priority to U.S. Provisional Application No. 63/546,084 filed Oct. 27, 2023, which is incorporated herein by reference in its entirety.

### BACKGROUND

### Field

The present disclosure relates generally to a nasal spray composition, and more particularly to a nasal composition and method that may be used in the prevention or treatment of respiratory tract infections.

### Background

In view of the bacterial and viral infections in the nasal passageway, the search for potential protective and therapeutic antibacterial strategies is of particular and urgent interest.

Therefore, a nasal spray composition effective in reducing viable pathogens and, more particularly, effective as preventive and adjuvant therapy for nasal infections or diseases caused by other pathogens (e.g., virus, bacteria), is desired.

### SUMMARY

The following is a non-exhaustive listing of some aspects of the present techniques of this disclosure. These and other aspects are described in this disclosure.

Some aspects include an antibacterial nasal composition effective in reducing viable bacteria, and, more particularly, providing a protective effect as preventive and adjuvant therapy for nasal infections or diseases caused by pathogens and viruses.

Some aspects include an antiviral nasal composition effective in reducing viable virus, and, more particularly, providing a protective effect as preventive and adjuvant therapy for NASAL INFECTIONS or respiratory diseases caused by other viruses.

Some aspects include an antibacterial nasal composition effective in reducing viable bacteria, and, more particularly, providing a protective effect as preventive and adjuvant therapy for respiratory diseases caused by bacteria.

Some aspects include an antibacterial nasal composition, containing thymol compounds (e.g., effective in modulating antiviral and antibacterial immunity and regulate inflammatory response, through combination with select metals ions such as zinc and magnesium. Some aspects include an antibacterial nasal composition containing hydrogen peroxide, effective in modulating antibacterial immunity. Some aspects include an antibacterial nasal composition containing iodine, effective in modulating antibacterial immunity and regulating an inflammatory response. Some aspects include an aerosol nasal composition containing aromatic compounds, effective in modulating antiviral and antibacterial immunity and regulating an inflammatory response.

Several inventive embodiments of the present disclosure are described below.

### BRIEF DESCRIPTION OF THE DRAWINGS

The above-mentioned aspects and other aspects of the present techniques will be better understood when the present application is read in view of the following FIGURE in which like numbers indicate similar or identical elements:

FIG. 1 is a flow diagram showing a method of use of a nasal spray, in accordance with some embodiments of the present disclosure. FIG. 1 depicts a method 100 of use, in accordance with some embodiments, and may generally include obtaining the ingredients 10 (e.g. chemicals and components, like those discussed elsewhere herein), in accordance with some embodiments of the present disclosure, as shown in block 10, mixing these ingredients to obtain an antibacterial nasal composition (e.g. nasal spray or nasal drop) as shown in block 12, packaging the antibacterial nasal composition, to facilitate shipment, prolong shelf-life, case of storage and facilitate administration, as shown in block 14, and nasally administering antibacterial nasal composition to an individual (e.g. with a nasal spray) as shown in block 16.

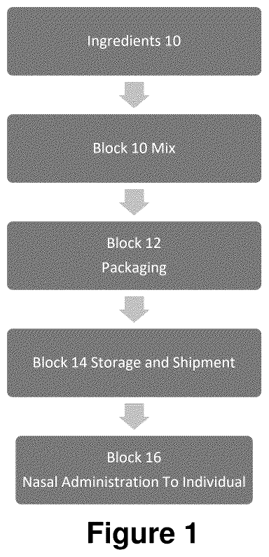

While the present techniques are susceptible to various modifications and alternative forms, specific embodiments thereof are shown by way of example in the drawings and will herein be described in detail. The drawings may not be to scale. It should be understood, however, that the drawings and detailed description thereto are not intended to limit the present techniques to the particular form disclosed, but to the contrary, the intention is to cover all modifications, equivalents, and alternatives falling within the spirit and scope of the present techniques as defined by the appended claims.

### DETAILED DESCRIPTION

To mitigate the problems described herein, this disclosure provides novel solutions and, in some cases just as importantly, recognize problems overlooked (or not yet foreseen) by others in prevention and treatment of bacterial and viral diseases in humans and animals. Further, because multiple problems are addressed, it should be understood that some embodiments are problem-specific, and not all embodiments address every problem with traditional systems described herein or provide every benefit described herein. That said, improvements that solve various permutations of these problems are described below.

### Definitions

The term “effective amount,” as used herein, refers to the amount of active agent needed to achieve the desired therapeutic or prophylactic effect, such as an amount that is sufficient to reduce pathogen (e.g., bacteria, virus) burden, reduce symptoms (e.g., fever, coughing, sneezing, nasal discharge, and the like), reduce occurrence of infection, reduce viral replication, or improve or prevent deterioration of respiratory function, produce an effective serum concentration of a pharmaceutically active agent, increase mucociliary clearance, reduce total inflammatory cell count, or modulate the profile of inflammatory cell counts. The actual effective amount for a particular use can vary according to the particular nasal composition, the mode of administration, and the age, weight, general health of the subject, and severity of the symptoms or condition being treated. Suitable amounts of nasal composition to be administered, and dosage schedules for a particular subject can be determined by a clinician of ordinary skill based on these and other considerations.

The term “pharmaceutically acceptable excipient” as used herein means that the excipient can be taken into the nostrils of the nose with no significant adverse toxicological effects on the mucous membranes of the nasal passage or nasal cavity of the subject. Such excipients are generally regarded as safe (GRAS) by the U.S. Food and Drug Administration.

### Nasal Formulations

In some embodiments, a nasal composition is formulated (or adapted) for preventing or treating viral infection through nasal passages, such as in a form of nasal drops, nasal washes, nasal packing, nasal sprays, and aerosol products.

In some embodiments, the nasal composition may have a viscosity in the range of 5 to 200,000 centipoise. The viscosity of the composition is important because it facilitates maintenance of the composition in the nasal cavity in contact with the nasal membrane or with mucous on the membrane. When the viscosity is less than about 2,500 centipoise, the composition tends to be drawn by gravity out of the nasal cavity. If the viscosity is in excess of about 40,000 centipoise, the thickness of the composition interferes with the diffusion of the chemicals through the composition to the nasal membrane.

In some embodiments, the nasal composition may include one or more pharmaceutically useful excipients, including pH adjusting agents, pH buffer, viscosity modifiers, osmotic agents, flavor, carbohydrate (e.g., to act as a sweetener), preservatives (e.g., metal chelators), adhesives and colorants. The selection and use of each agent are determined based on the practices known to those skilled in art.

In some embodiments, the nasal composition may further include a buffering agent, for example, a sodium phosphate buffer (e.g., sodium dihydrogen phosphate and disodium hydrogen phosphate).

In some embodiments, the nasal composition may further include one or more surfactants, for example selected from the group consisting of anionic, cationic, zwitterionic, and nonionic surfactants, and mixtures thereof.

In some embodiments, the nasal composition may further include a thickening agent, such as xanthan gum or carrageenan, vermiculite thickener, carbomers, and combinations thereof.

In some embodiments, the pH of the nasal composition is from about pH 2 to about pH 10. In some embodiments, the pH of the nasal composition is from about pH 2 to about pH 8. In some embodiments, the pH of the nasal composition is from about pH 4 to about pH 8. In some embodiments, the pH of the nasal composition is from about pH 4 to about pH 10. The pH of the nasal composition may be about 2, about 3, about 4, about 5, about 6, about 7, about 8, about 9, or about 10. In some embodiments, the pH of the nasal composition is about pH 6. The preferred pH of the formulation can differ depending on the specific bacterial indication that is being treated. For example, the pH needed to effectively kill a particular bacteria may be more or less acidic than the pH needed to effectively kill a different bacteria.

In one embodiment, the nasal composition comprises water, sodium chloride, sodium phosphate dibasic, potassium chloride, thymol, calcium chloride dihydrate, aloe vera, sodium ascorbate, ascorbic acid, and magnesium chloride hexahydrate.

In some embodiments the nasal composition comprises water, sodium chloride, sodium phosphate dibasic, potassium citrate, thymol, calcium chloride dihydrate, magnesium chloride hexahydrate, benalkonium chloride, and citric acid.

In some embodiments, the nasal composition may be selected from the group consisting of water, Sodium Chloride, Sodium Phosphate Dibasic, Potassium Citrate, Thymol, Calcium Chloride Dihydrate, Magnesium Chloride Hexahydrate, Benzalkonium Chloride (50%), and Citric Acid.

In some embodiments, the nasal composition may be selected from the group consisting of water, Sodium Chloride, Sodium Phosphate Dibasic, Potassium Citrate, Thymol, Calcium Chloride Dihydrate, Magnesium Chloride Hexahydrate, iodine, and Citric Acid.

The amount of water in the nasal composition may be about 60% to about 98% of the nasal composition by weight, for example the amount of water in the nasal composition may be about 60%, 65%, 70%, 72%, 74%, 76%, 78%, 80%, 82%, 84%, 86%, 88%, 90%, 95%, or 98% of the nasal composition by weight.

The amount of potassium chloride in the nasal composition may be about 0.5% to about 2% of the nasal composition by weight, for example, the amount of potassium chloride in the nasal composition may be about 0.5%, 0.6%, 0.7%, 0.8%, 0.9%, 1.0%, 1.2%, 1.4%, 1.6%, 1.8%, or 2% of the nasal composition by weight.

The amount of thymol in the nasal composition may be about 0.004% to about 0.049% of the nasal composition by weight, for example, the amount of rubidium chloride in the nasal composition may be about 0.004%, 0.005%, 0.006%, 0.008%, 0.009%, 0.010%, 0.012%, 0.014%, 0.016%, 0.020%, 0.025%, 0.030%, 0.035%, 0.040%, 0.045%, 0.049% of the nasal composition by weight.

The amount of calcium chloride dihydrate in the nasal composition may be about 0.0001% to about 0.0005% of the nasal composition by weight, for example, the amount of calcium chloride dihydrate in the nasal composition may be about 0.0001%, 0.00015%, 0.00020%, 0.00022%, 0.00023%, 0.00025%, 0.00030%, 0.00040%, or 0.00050% of the nasal composition by weight.

The amount of iodine in the nasal composition may be about 0.00010% to about 0.020% of the nasal composition by weight, for example, the amount of zinc chloride in the nasal composition may be about 0.00010%, 0.00011%, 0.00012%, 0.00013%, 0.00014%, 0.00015%, 0.00016%, 0.00017%, 0.00018%, to 0.019%, or 0.020% of the nasal composition by weight.

The amount of hydrogen peroxide in the nasal composition may be about 0.1% to about 2% of the nasal composition by weight, for example, the amount of potassium chloride in the nasal composition may be about 0.1%, 0.2%, 0.3%, 0.4%, 0.5%, 0.6%, 0.7%, 0.8%, 0.9%, 1.0%, 1.2%, 1.4%, 1.6%, 1.8%, or 2% of the nasal composition by weight.

### Methods of Treatment

The disclosure also relates to methods of treating and preventing respiratory diseases (e.g., respiratory disease caused by viral and/or bacterial infection). The nasal compositions described herein can be administered to a subject in need thereof for the treatment of respiratory diseases, such as respiratory caused by viral infections (e.g., coronavirus, influenza virus, parainfluenza virus, respiratory syncytial virus, rhinovirus, adenovirus, metapneumovirus, coxsackie virus, echo virus, herpes virus, cytomegalovirus, and the like), or bacterial infections (e.g., Streptococcus pneumoniae, which is commonly referred to as pneumococcus, Staphylococcus aureus, Burkholderis ssp., Streptococcus agalactiae, Haemophilus influenzae, Haemophilus parainfluenzae, Klebsiella pneumoniae, Escherichia coli, Pseudomonas aeruginosa, Moraxella catarrhalis, Chlamydophila pneumoniae, Mycoplasma pneumoniae, Legionella pneumophila, Serratia marcescens, Mycobacterium tuberculosis, Bordetella pertussis, and the like).

In a particular aspect, the nasal compositions described herein are administered to a subject in thereof for treatment or prevention of the common cold (e.g., a rhinovirus), a COVID19 infection (e.g, caused by SARS-COV-2 or a variant thereof), or the flu (e.g., influenza virus).

In a particular aspect, the nasal compositions described herein are administered to a subject in need thereof for treatment or prevention of allergies, hay fever, and other nasal related inflammation.

In some embodiments, nasally administering or nasal administration includes administering the compositions into nostrils of the nose to the mucous membranes of the nasal passage or nasal cavity of the subject. Such formulations may be administered, for example, as a nasal spray, nasal inhaler, nasal drop, aerosol, propellants, pressured dispersion, aqueous aerosol, nebulizer, nasal suspension, instillation, nasal gel, nasal ointment and nasal cream by aid of any new or old type device. Administration of compositions of the present disclosure may also take place using a nasal tampon or nasal sponge containing the compositions.

In some embodiments, the nasal composition may be delivered using a nasal device that supplies a fixed (measured) volume or an amount (dose) of a nasal composition after each actuation. Examples of measured dose devices for nasal administration include an atomizer, sprayer, dropper, squeeze tube, squeeze spray bottle, pipette, blister, nasal cannula, measured dose device, spray inhaler nasal, bi-directional breathing-powered delivery device, pump sprayer, pre-compression measured dose spray pump, monopulverization pump, bipulverization pump and pressurized measured dose device. The administration device may be a disposable single-dose device, reusable single-dose device, disposable multi-dose device or reusable multi-dose device.

Suitable dosing to provide the desired therapeutic effect can be determined by a clinician based on the severity of the condition (e.g., infection), overall well-being of the subject and the subject's tolerance to nasal compositions and other considerations. Based on these and other considerations, a clinician can determine appropriate doses and intervals between doses. Generally, nasal compositions are administered once, twice or three times a day, as needed.

Formulations may be conveniently presented in unit dosage form and may be prepared by any methods known in the art.

In some embodiments, the nasal composition may decrease the concentration of a bacteria by more than 90%, 95%, or 99% after an hour incubation.

In some embodiments, the nasal composition may decrease the concentration of a bacteria by more than 90%, 95%, or 99% after less than 1, 5, 10, 30, or 60 minutes incubation.

Exemplification is presented below that describes a preliminary in vitro study on antibacterial activity of the nasal compositions with the present techniques.

It should be understood that the description and the figures are not intended to limit the present techniques to the particular form disclosed, but to the contrary, the intention is to cover all modifications, equivalents, and alternatives falling within the spirit and scope of the present techniques as defined by the appended claims. Further modifications and alternative embodiments of various aspects of the techniques will be apparent to those skilled in the art in view of this description. Accordingly, this description and the drawings are to be construed as illustrative only and are for the purpose of teaching those skilled in the art the general manner of carrying out the present techniques. It is to be understood that the forms of the present techniques shown and described herein are to be taken as examples of embodiments. Elements and materials may be substituted for those illustrated and described herein, parts and processes may be reversed or omitted, and certain features of the present techniques may be utilized independently, all as would be apparent to one skilled in the art after having the benefit of this description of the present techniques. Changes may be made in the elements described herein without departing from the spirit and scope of the present techniques as described in the following claims. Headings used herein are for organizational purposes only and are not meant to be used to limit the scope of the description.

As used throughout this application, the word “may” is used in a permissive sense (i.e., meaning having the potential to), rather than the mandatory sense (i.e., meaning must). The words “include”, “including”, and “includes” and the like mean including, but not limited to. As used throughout this application, the singular forms “a,” “an,” and “the” include plural referents unless the content explicitly indicates otherwise. The term “or” is, unless indicated otherwise, non-exclusive, i.e., encompassing both “and” and “or.” Terms describing conditional relationships, e.g., “in response to X, Y,” “upon X, Y,”, “if X, Y,” “when X, Y,” and the like, encompass causal relationships in which the antecedent is a necessary causal condition, the antecedent is a sufficient causal condition, or the antecedent is a contributory causal condition of the consequent, e.g., “state X occurs upon condition Y obtaining” is generic to “X occurs solely upon Y” and “X occurs upon Y and Z.” Such conditional relationships are not limited to consequences that instantly follow the antecedent obtaining, as some consequences may be delayed, and in conditional statements, antecedents are connected to their consequents, e.g., the antecedent is relevant to the likelihood of the consequent occurring. Statements in which a plurality of attributes or functions are mapped to a plurality of objects (e.g., one or more processors performing steps A, B, C, and D) encompasses both all such attributes or functions being mapped to all such objects and subsets of the attributes or functions being mapped to subsets of the attributes or functions (e.g., both all processors each performing steps A-D, and a case in which processor 1 performs step A, processor 2 performs step B and part of step C, and processor 3 performs part of step C and step D), unless otherwise indicated. Further, unless otherwise indicated, statements that one value or action is “based on” another condition or value encompass both instances in which the condition or value is the sole factor and instances in which the condition or value is one factor among a plurality of factors. Unless otherwise indicated, statements that “each” instance of some collection have some property should not be read to exclude cases where some otherwise identical or similar members of a larger collection do not have the property, i.e., each does not necessarily mean each and every. Limitations as to sequence of recited steps should not be read into the claims unless explicitly specified, e.g., with explicit language like “after performing X, performing Y,” in contrast to statements that might be improperly argued to imply sequence limitations, like “performing X on items, performing Y on the X′ed items,” used for purposes of making claims more readable rather than specifying sequence. Statements referring to “at least Z of A, B, and C,” and the like (e.g., “at least Z of A, B, or C”), refer to at least Z of the listed categories (A, B, and C) and do not require at least Z units in each category. Unless specifically stated otherwise, as apparent from the discussion, it is appreciated that throughout this specification discussions utilizing terms such as “processing,” “computing,” “calculating,” “determining” or the like refer to actions or processes of a specific apparatus, such as a special purpose computer or a similar special purpose electronic processing/computing device. The terms “first”, “second”, “third,” “given” and so on, if used in the claims, are used to distinguish or otherwise identify, and not to show a sequential or numerical limitation.

1. A nasal composition comprising one or more phenolic compounds in an amount effective to modulate antiviral and/or antibacterial immunity.

2. The nasal composition of claim 1, wherein the nasal composition has a viscosity between 5 to 1000 centipoise.

3. The nasal composition claim 1, wherein the nasal composition comprises water, sodium chloride, sodium phosphate dibasic, potassium chloride, thymol, hydrogen peroxide, iodine, aloe vera, and ascorbic acid.

4. The nasal composition of claim 3, wherein the thymol is present in an amount from about 0.0001% to about 0.05% of the total weight of the nasal composition.

5. The nasal composition of claim 1, further comprising an excipient.

6. The nasal composition of claim 1, further comprising a buffering agent.

7. The nasal composition of claim 1, further comprising an anti-inflammatory and antioxidant agent.

8. The nasal composition of claim 1, further comprising a thickening agent.

9. The nasal composition of claim 1, wherein a pH of the nasal composition is from about pH2 to about pH10.

10. The nasal composition of claim 1, wherein the nasal composition is configured for delivery as nasal spray, nasal gel, nasal ointment, nasal cream, or aerosol.

11. A method of treating a respiratory tract infection comprising administering to nasal passages of a subject in need thereof an effective amount of the nasal composition comprising one or more phenolic compounds in an amount effective to modulate antiviral and/or antibacterial immunity.

12. The method of claim 11, wherein the respiratory tract infection is a viral infection or a bacterial infection.

13. The method of claim 12, wherein the respiratory tract infection is a viral infection.

14. The method of claim 12, wherein the respiratory tract infection is a bacterial infection.

15. A method of treating or preventing nasal infections, the method comprising administering an effective amount of the nasal composition comprising one or more phenolic compounds in an amount effective to modulate antiviral and/or antibacterial immunity.

16. The method of claim 15, further comprising decreasing bacterial concentration in a bacteria stock of bacteria comprising incubating the bacterial stock with the nasal composition for a time period of 60 minutes or less.

17. The method of claim 16, wherein the time period is 30 minutes or less.

18. The method of claim 16, wherein the time period is 10 minutes or less.

19. The method of claim 16, wherein the time period is 5 minutes or less.

20. The method of claim 16, wherein the time period is 1 minute or less.
